(19) United States
(12) Patent Application Publication
(10) Pub. No.: US 2010/0055769 A1
(43) Pub. Date: Mar. 4, 2010

(54) BIOTECHNOLOGICAL DEVICE INCLUDING A STRUCTURED HYDROGEL PERMEATION LAYER

(75) Inventors: Ralph Kurt, Eindhoven (NL); Dick Jan Broer, Geldrop (NL); Roel Penterman, Eindhoven (NL); Emiel Peeters, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/517,268

(22) PCT Filed: Nov. 29, 2007
(86) PCT No.: PCT/IB07/54836

§ 371 Date: Jun. 2, 2009

(30) Foreign Application Priority Data
Dec. 4, 2006 (EP) 06125327.4

(51) Int. Cl. C12M 1/34; B01J 8/00; B01J 8/18; C12M 1/00; G01N 30/96; C08F 2/46

(52) U.S. Cl. 435/287.2; 422/240; 422/139; 435/283.1; 435/287.1; 422/69; 522/174; 522/175; 522/167; 522/104

(57) ABSTRACT

The present invention relates to a biotechnological device including a responsive hydrogel permeation layer with an intrinsic structural gradient.

The present invention is directed to the field of biotechnological devices.

In recent years, several biotechnological devices, e.g. analytical biotechnological devices have been described which use permeation layers. The permeation layer e.g. provides capture sites for the immobilization of nucleic acids (or other target molecules). In these devices main function of the permeation layer is to separate the captured target molecules from the highly reactive electrochemical environment generated immediately at the electrode surface. The layer also allows ions and gasses arising from electrochemical reactions at the electrodes to gradually diffuse into the biological solution.

E.g. from the U.S. Pat. No. 6,960,298 and related patents cited therein, which are hereby incorporated by reference, synthetic polymer hydrogel permeation layers are known for use on active electronic matrix devices for biological assays.

Most types of permeation layers used in the field are hydrogel layers, especially due to their ability to shrink and/or swell. In many applications it is even possible to shrink and/or swell permeation layers upon external stimuli, e.g. heating/cooling or a change in pH, applying an electric field, charge to or a current through at least parts of said permeation layer.

However, in many biotechnological devices—may it be analysis devices or devices for drug release—using such permeation layers in the field, an “enclosure” phenomenon (also called the “thick skin” phenomenon) is a common problem when seeking to increase the sensitivity and efficacy of the device. Such an enclosure occurs e.g. when the upper hydrogel layer shrinks so fast that molecules which are located in the lower part of the layer cannot leave the permeation layer any more or not fast enough and are therefore “trapped” or “enclosed” in the layer, which may lead to maldetection and decreasing sensitivity of the device. Moreover the speed of such devices is limited.

It is therefore an object of the present invention to provide a device, which is able of at least partially overcoming some of the above-mentioned drawbacks and helps to increase the specifity, the speed, and/or the effectivity of the analyis.

This object is solved by a device according to claim 1 of the present invention. Accordingly, a biotechnological device is provided, comprising a responsive hydrogel permeation layer which has at least in parts of the permeation layer an intrinsic structural gradient in the direction of the layer thickness and/ or in a direction substantially parallel to a flow of at least a predefined species of biomolecules.

The term “biotechnological device” is to be understood in its widest sense and includes especially one or more of the following devices:
devices for the detection of one or more target molecules in a fluid sample, especially devices for the detection of biomolecules in aqueous solution.
devices for the controlled release of a compound, especially for drug release.
devices for performing amplification reactions such as PCR (polymerase chain reaction), QPCR (quantitative PCR), RTPCR (real time PCR).
artificial scaffolds for tissue engineering and (stem) cell therapies, including devices for the release of molecules such as growth factors, cytokines etc. to stimulate growth or proliferation of cells and devices which pump nutrients towards cells or accelerate degradation of the scaffold on command.

The terms “biomolecules” as well as “target molecules”, “capture sites” “drugs” according to the present invention are to be understood in the widest sense and especially include and/or mean the product(s) of an amplification reaction, including both target and signal amplification); purified samples, such as purified genomic DNA, RNA, proteins, etc.; raw samples (bacteria, virus, genomic DNA, etc.); biological molecular compounds such as, but not limited to, nucleic acids and related compounds (e.g. DNAs, RNAs, oligonucleotides or analogs thereof, PCR products, genomic DNA, bacterial artificial chromosomes, plasmids and the like), proteins and related compounds (e.g. polypeptides, peptides, monoclonal or polyclonal antibodies, soluble or bound receptors, transcription factors, and the like), antigens, ligands, haptens, carbohydrates and related compounds (e.g. polysaccharides, oligosaccharides and the like), cellular fragments such as membrane fragments, cellular organelles, intact cells, bacteria, viruses, protozoa, and the like.

By using such a device at least one or more of the following advantages can be achieved for a wide range of applications within the present invention
Due to the possibility to control the shrinking/swelling process of the hydrogel permeation layer in more great detail, the described “enclosure” phenomena can be reduced or in some application be avoided at all.
The design of the device can be made more compact.
The flow of fluids through the permeation layer can be significantly accelerated for a wide range of applications within the present invention.
fast, efficient and well controlled release of molecules/agents such as drug
effective fluid manipulation e.g. washing.

The term “hydrogel” in the sense of the present invention especially means that at least a part of the permeation layer comprises polymers that in water form a water-swollen network and/or a network of polymer chains that are water-soluble. Preferably the hydrogel permeation layer comprises in swollen state ≧50% water and/or solvent, more preferably ≧70% and most preferred ≧90%, whereby preferred solvents include organic solvents, preferably organic polar solvents and most preferred alkanols such as Ethanol, Methanol and/or (Iso-) Propanol.

In the sense of the present invention, the term “responsive” means and/or includes especially that the hydrogel permeation layer is responsive in such a way that it displays a change of shape and total volume upon a change of a specific parameter. Such parameter can be a physical (temperature, pressure) or chemical property (ionic concentration, pH, analyte concentration) or biochemical property (enzymatic activity).

The term “in the direction of the layer thickness” does not mean that the intrinsic structural gradient is present in the direction of the layer thickness only. This may be an embodiment of the present invention, however, according to a further embodiment of the present invention (as will be described later on) there may be an intrinsic structural gradient in further directions, too.

According to an embodiment of the present invention, the device comprises a hydrogel permeation layer which is responsive to at least one external stimulus, upon which the flow of at the least a predefined species of biomolecules is altered.

The term “external” especially means that the hydrogel permeation layer is triggered by a means and/or stimulus provided and/or arising outside the layer, such as a change in pH or temperature, however it is clear to any skilled person in the art that this means and/or stimulus might arise from an actuation means inside the device, such as a heater etc.

The stimuli preferably include—but not limited to—physical stimuli (temperature, pressure, voltage, current, charge), chemical stimuli (ionic concentration, pH, analyte concentration) or biochemical stimuli (enzymatic activity, presence or absence of analyte).

According to an embodiment of the present invention, the device comprises a hydrogel permeation layer which comprises at least one supporting structure.

The term “supporting structure” in the sense of the present invention means and/or includes—but not limited to—supporting substrate(s) (either flat or curved, closed for fluid flow or permeable, porous membrane or a mesh like structure) underneath as well as supporting structure(s) as part of the layer, such as rigid or elastic bars, wires, walls etc. or said supporting structure may form compartments, reservoirs, cavities or channels. Said supporting structure comprises rigid materials selected from group—but not limited to—glass, silicon, metal, metal oxides, polymeric material (such as PVC, polyimide, PC, but also organic resist material such as SU-8 and the like).

According to an embodiment of the present invention, the hydrogel material comprises a material selected out of the group comprising poly(meth)acrylic materials, substituted vinyl materials or mixture thereof.

According to an embodiment of the present invention, the hydrogel material comprises a poly(meth)acrylic material made out of the polymerization of at least one (meth)acrylic monomer and at least one polyfunctional (meth)acrylic monomer.

According to an embodiment of the present invention, the (meth)acrylic monomer is chosen out of the group comprising (meth)acrylamide, hydroxyethyl(meth)acrylate, ethoxyethoxyethyl(meth)acrylate or mixtures thereof.

According to an embodiment of the present invention, the polyfunctional (meth)acrylic monomer is a bis-(meth)acryl and/or a tri-(meth)acryl and/or a tetra-(meth)acryl and/or a penta-(meth)acryl monomer.

According to an embodiment of the present invention, the polyfunctional (meth)acrylic monomer is chosen out of the group comprising bis(meth)acrylamide, tripropyleneglycol di(meth)acrylates, pentaerythritol tri(meth)acrylate polyethyleneglycoldi(meth)acrylate, ethoxylated bisphenol-A-di(meth)acrylate, hexanedioldi(meth)acrylate or mixtures thereof.

According to an embodiment of the present invention, the hydrogel material comprises an anionic poly(meth)acrylic material, preferably selected out of the group comprising (meth)acrylic acids, arylsulfonic acids, especially styrenesulfonic acid, itaconic acid, crotonic acid, sulfonamides or mixtures thereof, and/or a cationic poly(meth)acrylic material, preferably selected out of the group comprising vinyl pyridine, vinyl imidazole, aminoethyl (meth)acrylates or mixtures thereof, co-polymerized with at least one monomer selected out of the group neutral monomers, preferably selected out of the group vinyl acetate, hydroxyethyl (meth)acrylate (meth)acrylamide, ethoxyethoxyethyl(meth)acrylate or mixture thereof, or mixtures thereof.

It is known for a wide range of these co-polymers to change their shape as a function of pH and to respond to an applied electrical field and/or current. Therefore these materials may be of use for a wide range of applications within the present invention.

According to an embodiment of the present invention, the hydrogel material comprises a substituted vinyl material, preferably vinylcaprolactam and/or substituted vinylcaprolactam.

According to an embodiment of the present invention, the hydrogel material is based on thermo-responsive monomers selected out of the group comprising N-isopropylamide, diethylacrylamide, carboxyisopropylacrylamide, hydroxymethylpropylmethacrylamide, acryloylalkylpiperazine and copolymers thereof with monomers selected out of the group hydrophilic monomers, comprising hydroxyethyl(meth)acrylate, (meth)acrylic acid, acrylamide, polyethyleneglycol(meth)acrylate or mixtures thereof, and/or co-polymerized with monomers selected out of the group hydrophobic monomers, comprising (iso)butyl(meth)acrylate, methylmethacrylate, isobomyl(meth)acrylate or mixtures thereof. These co-polymers are known to be thermo-responsive and therefore may be of use for a wide range of applications within the present invention.

According to an embodiment of the present invention, the hydrogel material is functionalized with reactive side-groups such as amines or active esters, especially to perform in-situ ‘cross-linking’ of DNA or anti-bodies.

According to an embodiment of the present invention, the structural gradient may comprise a gradient in at least one or more of the following features:

composition, concentration, porosity, pore size, pore size distribution, pore interconnectivity, ordering parameter, cross-link density, charge density, polarity, pKₐ, LCST transition temperature, density, permeability.

It should be noted that the structural gradient according to the invention is present (and measurable) in an equilibrium state, i.e. not only as a transient effect caused by kinetic effects. However said structural gradient may be present in a swollen state (only), in a shrunken state (only), in an at least partially swollen intermediate state (only) as well as in more than one state or in all these states.

It should be noted that said structural gradient should be understood in the widest sense and may be realized—but not limited to—as a substantially linear gradient, and/or as a non-linear gradient and/or a substantially stepwise gradient (comprising more than one step).

According to an embodiment of the present invention, the device comprises at least one capture site, whereby the permeation layer has an intrinsic structural gradient in regions of the permeation layer which are associated with the at least one capture site.

According to an embodiment of the present invention, the device comprises at least one drug release site, whereby the permeation layer has an intrinsic structural gradient in regions of the permeation layer which are associated with the at least one drug release site.

According to an embodiment of the present invention, the hydrogel permeation layer which is responsive to at least one external stimulus, upon which the flow of at the least a predefined species of biomolecules is altered in way that said flow is substantially in the direction of the gradient.

According to an embodiment of the present invention, the hydrogel permeation layer which is responsive to at least one external stimulus, upon which the flow of at the least a predefined species of biomolecules is altered in way that said flow is substantially in a direction towards the surface of said hydrogel permeation layer.

According to an embodiment of the present invention, the hydrogel permeation layer which is responsive to at least one external stimulus, upon which the flow of at the least a predefined species of biomolecules is altered in way that said flow is substantially in a direction from the surface of said hydrogel permeation layer (towards said substrate).

According to an embodiment of the present invention, the thickness of the hydrogel permeation layer is >1 μm and <1 mm, more preferably >5 μm and <500 μm, and most preferably >10 μm and <200 μm.

According to an embodiment of the present invention, the intrinsic structural gradient includes cross-link density and the cross-link density changes by a factor ≧1.5 over the thickness of the permeation layer. This has been shown to be advantageous for a wide range of applications within the present invention.

According to an embodiment of the present invention, the intrinsic structural gradient includes cross-link density and the cross-link density changes by a factor ≧2, more preferably ≧5, most preferred ≧10 over the thickness of the permeation layer.

According to an embodiment of the present invention, the permeation layer comprises at least one region, in which the cross-link density changes ≧20% per μm, preferably ≧25% per μm.

In the sense of the present invention, the term “crosslink density” means or includes especially the following definition: The crosslink density δₓ is here defined as

$${δ}_{X} = \frac{X}{L + X}$$

where X is the mole fraction of polyfunctional monomers and L the mole fraction of linear chain (=non polyfunctional) forming monomers. In a linear polymer δₓ=0, in a fully crosslinked system δₓ=1.

According to an embodiment of the present invention, the intrinsic structural gradient includes porosity and the porosity of the hydrogel permeation layer changes by a factor ≧1.2 over the thickness of the permeation layer. This has been shown to be advantageous for a wide range of applications within the present invention.

According to an embodiment of the present invention, the intrinsic structural gradient includes porosity and the porosity of the hydrogel permeation layer changes by a factor ≧1.5, more preferred ≧1. 8 and most preferred ≧2 over the thickness of the permeation layer.

According to an embodiment of the present invention, the permeation layer comprises at least one region, in which the porosity changes ≧20% per μm, preferably ≧25% per μm.

According to an embodiment of the present invention, the intrinsic structural gradient includes one out of the group pore size, pore size distribution, and permeability and at least one of these parameters changes by a factor ≧1.5 over the thickness of the permeation layer. According to an embodiment of the present invention, the permeation layer comprises at least one region, in which at least on of said parameters changes ≧20% per μm, preferably ≧25% per μm. This has been shown to be advantageous for a wide range of applications within the present invention.

According to an embodiment of the present invention, the intrinsic structural gradient includes one out of the group pore size, pore size distribution, and permeability and at least one of these parameters changes by a factor ≧2, more preferred ≧5 and most preferred ≧10 over the thickness of the permeation layer.

According to an embodiment of the present invention, the intrinsic structural gradient includes a change of composition in that way that along the gradient the concentration of a material A is increased whereby the concentration of a material B is decreased and/or along the gradient the concentration of a material A is decreased whereby the concentration of a material B is increased. As a consequence the fraction or the ratio of material A with respect to material B increases or decreases in the direction of said gradient, respectively.

It should be noted that by material A and B monomers, polymers, oligomers, or small molecules are included. In case that A and B are monomers, the gradient may be realized by a co-polymer including A and B as monomers. Therefore according to an embodiment of the present invention the permeation layer comprises co-polymers built up from monomers or oligomers A and B, whereby the ratio A/B decreases or increases in the direction of the gradient.

According to an embodiment of the present invention, the concentration of material A and of material B changes across the thickness of the layer by a factor ≧1.2. This has been shown to be advantageous for a wide range of applications within the present invention.

According to an embodiment of the present invention, the concentration of material A and of material B changes across the thickness of the layer by a factor ≧1.3, more preferred ≧1.5, and most preferred ≧2. According to an embodiment of the present invention, the permeation layer comprises at least one region, in which said concentration of material A and of material B changes ≧10%, preferably ≧15% per μm.

According to an embodiment of the present invention, the material A is selected out of a group comprising thermo-responsive monomers, preferably selected out of the group comprising N-isopropylamide, diethylacrylamide, carboxyisopropylacrylamide, hydroxymethylpropylmethacrylamide, acryloylalkylpiperazine, and copolymers thereof with monomers selected out of the group hydrophilic monomers, comprising hydroxyethyl(meth)acrylate, (meth)acrylic acid, acrylamide, polyethyleneglycol(meth)acrylate or mixtures thereof, and/or co-polymerized with monomers selected out of the group hydrophobic monomers, comprising (iso)butyl(meth)acrylate, methylmethacrylate, isobornyl(meth)acrylate or mixtures thereof.

According to an embodiment of the present invention, the material B is selected out of a group comprising hydrophilic monomers, preferably selected out of the group comprising hydroxymethacrylate, methacrylic acid or mixtures thereof and hydrophobic monomers, preferably selected out of the group (iso)butylmethacrylate, hexanedioldimethacrylate or mixtures thereof.

Preferably the monomers A and B are selected so that A has a different reactivity than B.

In case that A is a thermo-responsive monomer and B is a hydrophilic monomer, it has been shown for many applications within the present invention that a hydrogel permeation layer with a gradient in LCST can be achieved easily and effective.

According to an embodiment of the present invention, the material A is selected out of a group comprising anionic monomers, preferably selected out of the group comprising (meth)acrylic acids, arylsulfonic acids, especially styrenesulfonic acid, itaconic acid, crotonic acid, sulfonamides or mixtures thereof.

According to an embodiment of the present invention, the material B is selected out of a group comprising neutral monomers, preferably selected out of the group comprising vinyl acetate, hydroxyethyl (meth)acrylate (meth)acrylamide, ethoxyethoxyethyl(meth)acrylate or mixtures thereof, cationic monomers preferably selected out of the group comprising vinyl pyridine, vinyl imidazole, aminoethyl (meth)acrylates or mixtures thereof or mixtures thereof, or mixtures thereof.

Preferably the monomers A and B are selected so that A has a different reactivity than B.

In case that A is an anionic monomer and B is a neutral and/or cationic monomer, it has been shown for many applications within the present invention that a hydrogel permeation layer with a gradient in pKₐ can be achieved easily and effective.

According to an embodiment of the present invention, the hydrogel permeation layer comprises a gradient in absorption, especially in the wavelength area of ≧220 nm and ≦450 nm.

Preferably this absorption gradient is caused by a gradient in concentration of a photoabsorbing material, whereby the photoabsorbing material preferably has an extinction of ≧20000, preferably ≧25000 and one maximum between ≧220 nm and ≦450 nm, more preferred ≧280 nm and ≦400 nm and most preferred ≧320 nm and ≦360 nm.

Preferably the photoabsorbing material is an azo compound. One suitable example is e.g. 4,4′ di-(methacryloyloxyhexyloxy)-3-methylstilbene.

According to an embodiment of the present invention, the hydrogelic permeation layer has a gradient in the LCST (lower critical solution temperature) and the LCST changes over the layer thickness by ≧1K. This has proven itself in practice for a wide range of applications within the present invention.

According to an embodiment of the present invention, the hydrogelic permeation layer has a gradient in the LCST (lower critical solution temperature) and the LCST changes over the layer thickness by ≧2K, more preferred ≧5K and most preferred ≧10K.

According to an embodiment of the present invention, the hydrogelic permeation layer has a gradient in the pKₐ and the difference in pKₐ over the layer thickness is ≧0.5. This has proven itself in practice in a wide range of applications within the present invention, especially when charged biomolecules, such as DNA and/or peptides and proteins are involved.

According to an embodiment of the present invention, the hydrogelic permeation layer has a gradient in the pKₐ and the difference in pKₐ over the layer thickness is ≧1, more preferred ≧2 and most preferred ≧3.

According to an embodiment of the present invention, the pKₐ on the “lower side” is ≦7, preferably ≦5, more preferably ≦3. According to an embodiment of the present invention, the pKₐ on the “higher side” is ≧7, preferably ≧8, more preferably ≧9, and most preferably ≧10.

The present invention furthermore relates of a method of producing a hydrogel permeation layer for use in a device as described herein, comprising the steps of:
a) providing at least two monomeric materials, whereby the two monomeric materials have a different reactivity, and at least one UV-absorbing compound to induce an intensity gradient during polymerization
b) performing a photo-polymerization

Due to the difference in reactivity between the monomers used to make the hydrogel permeation layer, one component (A) will polymerize faster in the top of the layer than in the bottom, resulting in diffusion of at least one component. Said diffusion can be upwards, downwards, sideward or a combination of all directions. Moreover monomers of component (A) can diffuse towards the top region where the faster polymerization occurs, whereas monomers of component (B), and/or non-reactive component(s) diffuse downwards. But it can also occur that monomers or polymers formed from component (A) diffuse downwards and/or monomers of component (B), and/or non-reactive component(s) diffuse upwards. As a result a concentration gradient will be formed causing the intrinsic structural gradient.

The present invention furthermore relates of a method of producing a hydrogel permeation layer for use in a device as described herein, comprising the steps of:
a) providing at least two monomeric materials, whereby the two monomeric materials have a different diffusion constant
b) causing at least one of the monomeric materials to diffuse in the direction of the gradient
c) performing a polymerization

The present invention furthermore relates of a method of producing a hydrogel permeation layer for use in a device as described herein, comprising the steps of:
a) providing at least two monomeric materials, whereby the two monomeric materials have a different sizes, and at least one UV-absorbing compound to induce an intensity gradient during polymerization
b) performing a photo-polymerization

Due to the different sizes of the monomeric materials, their diffusion velocity will be different; thus the smaller molecule will be able to diffuse faster upwards, which causes a local increased concentration which then finally causes the intrinsic structural gradient.

A device according to the present invention may be of use in a broad variety of systems and/or applications, amongst them one or more of the following:
biosensors used for molecular diagnostics
rapid and sensitive detection of proteins and nucleic acids in complex biological mixtures such as e.g. blood or saliva
high throughput screening devices for chemistry, pharmaceuticals or molecular biology
testing devices e.g. for DNA or proteins e.g. in criminology, for on-site testing (in a hospital), for diagnostics in centralized laboratories or in scientific research
tools for DNA or protein diagnostics for cardiology, infectious disease and oncology, food, and environmental diagnostics
tools for combinatorial chemistry
analysis devices
drug delivery devices for medical applications
devices for the detection of one or more target molecules in a fluid sample, especially devices for the detection of biomolecules in aqueous solution.
devices for the controlled release of a compound, especially for drug release.
devices for performing amplification reactions such as PCR (polymerase chain reaction), QPCR (quantitative PCR), RTPCR (real time PCR).
artificial scaffolds for tissue engineering and (stem) cell therapies, including devices for the release of molecules such as growth factors, cytokines etc. to stimulate growth or proliferation of cells and devices which pump nutrients towards cells or accelerate degradation of the scaffold on command.

The aforementioned components, as well as the claimed components and the components to be used in accordance with the invention in the described embodiments, are not subject to any special exceptions with respect to their size, shape, material selection and technical concept such that the selection criteria known in the pertinent field can be applied without limitations.

### BRIEF DESCRIPTION OF THE DRAWINGS

Additional details, features, characteristics and advantages of the object of the invention are disclosed in the subclaims, the figures and the following description of the respective figures and examples, which—in an exemplary fashion—show several preferred embodiments of a device according to the invention.

FIG. 1 shows a very schematic cross-sectional partial view showing a device according to a first embodiment of the present invention with a hydrogel permeation layer which has an intrinsic structural gradient in the direction of the layer thickness only

FIG. 2 shows the device of FIG. 1 after shrinking of the permeation layer;

FIG. 3 shows a very schematic cross-sectional partial view showing a device according to a second embodiment of the present invention with a hydrogel permeation layer which has an intrinsic structural gradient associated with a capture site;

FIG. 4 shows a very schematic partial top view of a device according to a third embodiment of the present invention with a hydrogel permeation layer which has an intrinsic structural gradient associated with a capture site; and

FIG. 5 shows a very schematic cross-sectional partial view showing a device according to a first embodiment of the present invention with a hydrogel permeation layer which has an intrinsic structural gradient in the direction of the flow of preselected biomolecules.

FIG. 6 shows the device of FIG. 5 after application of an external stimulus.

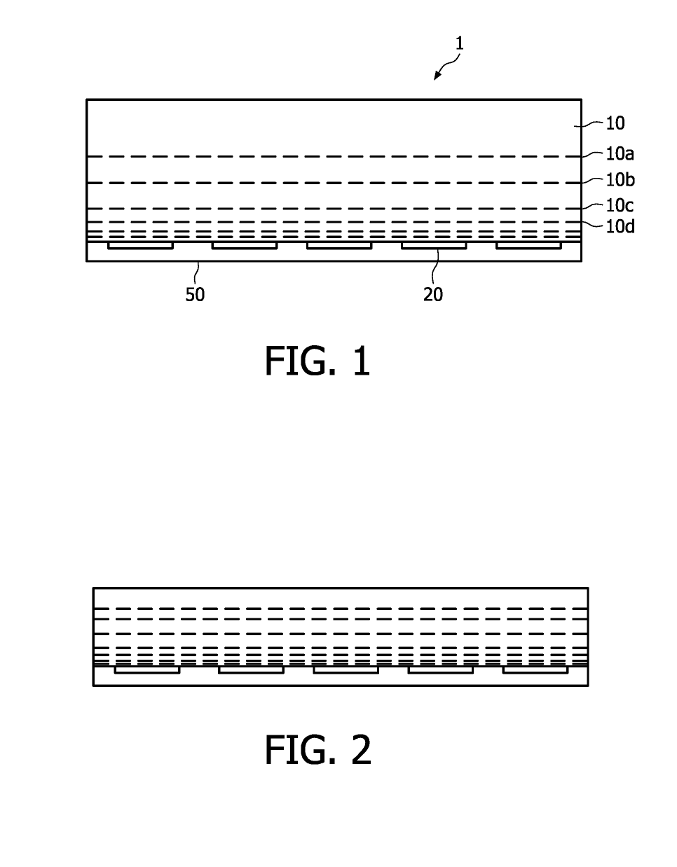

FIG. 1 shows a very schematic cross-sectional partial view showing a device 1 according to a first embodiment of the present invention with a hydrogel permeation layer 10 which has an intrinsic structural gradient in the direction of the layer thickness only. The device furthermore comprises several capture sites 20 (according to a different embodiment, these may be also drug release sites) provided with a substrate material 50.

As indicated by the dotted lines, some of which are referred to as 10a-d, there is provided a structural gradient (in this embodiment crosslink density) in the direction of the layer thickness. From every line, the crosslink density is increased by 5%—so that at line 10b the crosslink density is 5% higher than at 10a, whereas at line 10c the crosslink density is 5% higher than at line 10b and so on.

FIG. 2 shows the device after shrinking of the permeation layer. Due to the fact that the crosslink density decreases when “moving away” from the capture sites, an “enclosure” or “thick skin” phenomenon can be avoided to a great extent or be exluded after all.

It should be noted that a shrinking/swelling process as indicated by FIGS. 1 and 2 might be done in cycles (i.e. several times subsequently swelling and shrinking etc.). This has been shown advantageous for many applications within the present invention to help to prevent foam forming, which often occurs when “pumping” is involved, especially e.g. in cases when contact with air might disturb the chemistry of an detection or amplification.

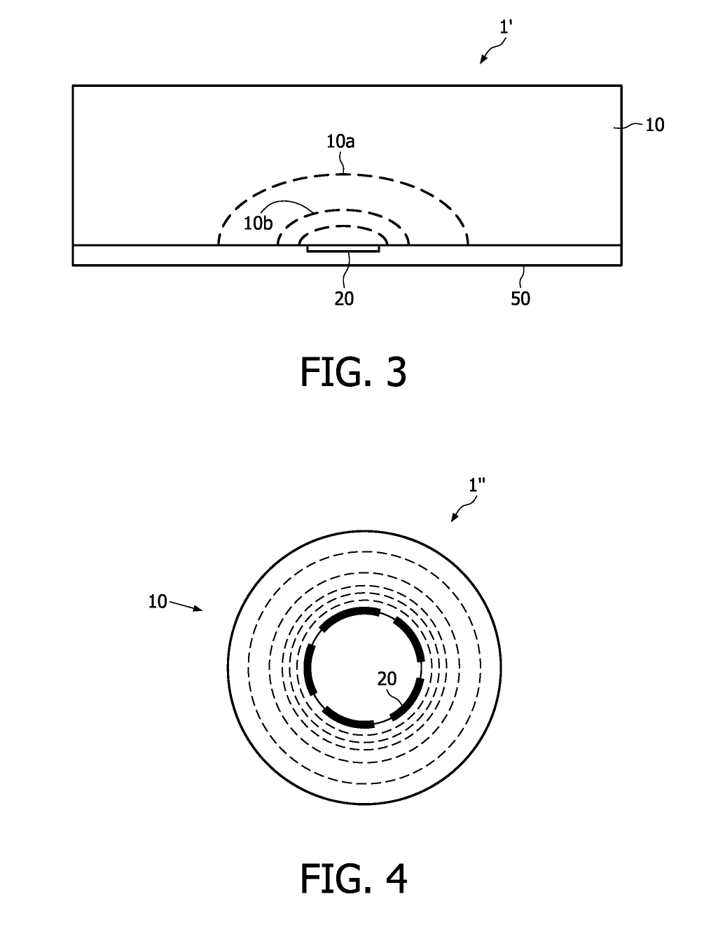

FIG. 3 shows a very schematic cross-sectional partial view showing a device 1′ according to a second embodiment of the present invention with a hydrogel permeation layer 10 which has an intrinsic structural gradient associated with a capture site 20. In an alternative embodiment (not shown in the figs), the capture site may be replaced by a drug delivery site.

In all drawings, for the sake of clearness and readability, the (essentially) same components and/or materials are referred to with the same numbers.

In this embodiment, too, the crosslink density is increased from lines 10a to 10b by 5% and so on (due to clarity reasons, only the first three niveaus are shown and only the first two are numbered).

The embodiment of FIG. 3 differs from that of FIGS. 1 and 2 in that the gradient is increased radially towards the capture site 20. This allows for a range of applications to even increase the efficacy of delivery and/or washing steps.

FIG. 4 shows a very schematic partial top view of a device 1″ according to a third embodiment of the present invention with a hydrogel permeation layer 10 which has an intrinsic structural gradient associated with a capture site 20. This embodiment differs from that of FIG. 3 in that the structural gradient is provided not “3D-radially” but in that the gradient is uniform in the direction of the layer thickness (which is perpendicular to the paper plane in FIG. 4) and extents itself “2D-radially” from the capture site 20.

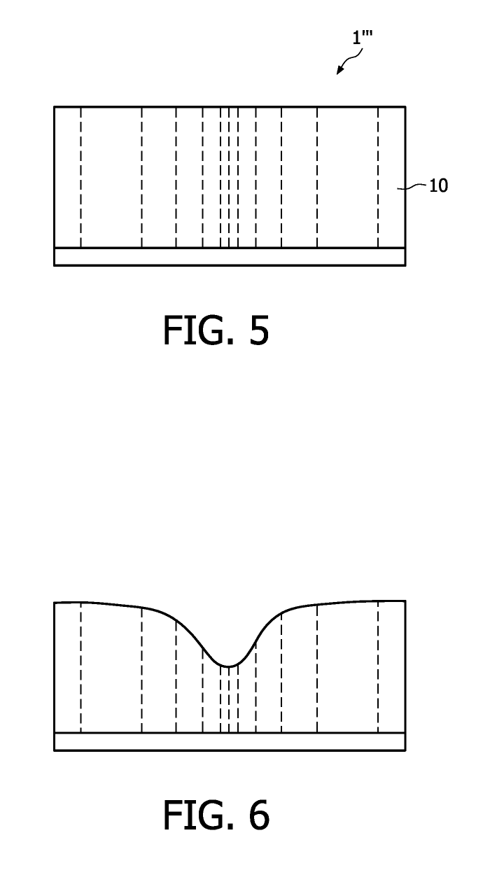

FIG. 5 shows a very schematic cross-sectional partial view showing a device 1′″ according to a first embodiment of the present invention with a hydrogel permeation layer which has an intrinsic structural gradient in the direction of the flow of preselected biomolecules. As can be seen from the dotted lines in FIG. 5, the gradient is somewhat “90°” to that of FIG. 1.

The gradient extents itself from the middle to the left and right, i.e. that in the middle the crosslink density is highest and decreases with 5% at every dotted line.

FIG. 6 shows the device of FIG. 5 after application of an external stimulus, which causes the hydrogel permeation layer to shrink according to the crosslink density. As can be seen from FIG. 6, the layer will then shrink in the middle section first, since there the density is highest, whereas the left and right sections will shrink less or may—according to the application—be unaffected.

The particular combinations of elements and features in the above detailed embodiments are exemplary only; the interchanging and substitution of these teachings with other teachings in this and the patents/applications incorporated by reference are also expressly contemplated. As those skilled in the art will recognize, variations, modifications, and other implementations of what is described herein can occur to those of ordinary skill in the art without departing from the spirit and the scope of the invention as claimed. Accordingly, the foregoing description is by way of example only and is not intended as limiting. The invention's scope is defined in the following claims and the equivalents thereto. Furthermore, reference signs used in the description and claims do not limit the scope of the invention as claimed.

1. A biotechnological device comprising a responsive hydrogelic permeation layer which has at least in parts of the permeation layer an intrinsic structural gradient in the direction of the layer thickness and/or in a direction substantially parallel to a flow of at least a predefined species of biomolecules.

2. A device according to claim 1 whereby the hydrogelic permeation layer is responsive to at least one of the stimuli comprising physical stimuli (temperature, pressure, voltage, current, charge), chemical stimuli (ionic concentration, pH, analyte concentration) and/or biochemical stimuli (enzymatic activity, presence or absence of analyte).

3. The device according to claim 1, whereby the structural gradient comprises a gradient in at least one or more of the following features: cross-link density, composition, porosity, ordering parameter, LCST (lower critical solution temperature).

4. The device according to claim 1, whereby the device comprises at least one capture and/or drug release site, whereby the permeation layer has an intrinsic structural gradient in regions of the permeation layer which are associated with the at least one capture site.

5. The device according to claim 1, whereby the device comprises at least one drug release site, whereby the permeation layer has an intrinsic structural gradient in regions of the permeation layer which are associated with the at least one drug release site.

6. The device according to claim 1, the intrinsic structural gradient includes cross-link density and the cross-link density changes by a factor ≧1.5 over the thickness of the permeation layer.

7. The device according to claim 1, the intrinsic structural gradient includes porosity and the porosity of the hydrogel permeation layer changes by a factor ≧1.2 over the thickness of the permeation layer.

8. The device according to claim 1, the hydrogelic permeation layer has a gradient in the LCST (lower critical solution temperature) and the LCST changes over the layer thickness by ≧1K.

9. A method of producing a hydrogel permeation layer according to claim 1, comprising the steps of:
(a) providing at least two monomeric materials, whereby the two monomeric materials have a different sizes and/or reactivities, and at least one UV-absorbing compound to induce an intensity gradient during polymerization
(b) performing a photo-polymerization

10. A system incorporating a device according to claim 1 and being used in one or more of the following applications:
biosensors used for molecular diagnostics
rapid and sensitive detection of proteins and nucleic acids in complex biological mixtures such as e.g. blood or saliva
high throughput screening devices for chemistry, pharmaceuticals or molecular biology
testing devices e.g. for DNA or proteins e.g. in criminology, for on-site testing (in a hospital), for diagnostics in centralized laboratories or in scientific research
tools for DNA or protein diagnostics for cardiology, infectious disease and oncology, food, and environmental diagnostics
tools for combinatorial chemistry
analysis devices
